(19) United States
(12) Patent Application Publication
(10) Pub. No.: US 2005/0070950 A1
(43) Pub. Date: Mar. 31, 2005

(54) Delivery assembly for use in surgery

(76) Inventors: Christopher Mason, Greater London (GB); Martin Arthur Town, Greater London (GB)

Correspondence Address: NIXON & VANDERHYE, PC, 1100 N GLEBE ROAD, 8TH FLOOR, ARLINGTON, VA, 22201-4714, US

(21) Appl. No.: 10/509,090

(22) PCT Filed: Mar. 28, 2003
(86) PCT No.: PCT/GB03/01368

(30) Foreign Application Priority Data
Mar. 28, 2002 (GB) 02074169

(51) Int. Cl. A61M 29/00

(52) U.S. Cl. 606/194

(57) ABSTRACT

A delivery assembly for use in surgery particularly laparoscopic surgery, comprises a surgically implantable construct containing viable cells, a support on which the construct is removably carried and holding means such as a vacuum system releasably holding the construct on the support. The support is selected from a tube having an interior surface on which the construct is held, and a rod having an exterior surface on which the construct is held. The support protects the delicate construct and maintains it in a desired position for surgical manipulation.

### FIELD OF THE INVENTION

This invention relates to a delivery assembly for use in surgery, which is applicable in any surgical technique practised on a human or animal body, and in particular in robot-controlled laparoscopic techniques.

### BACKGROUND OF THE INVENTION

Many surgical techniques involve the implantation of a surgically implantable construct, for example a replacement blood vessel in coronary artery bypass surgery. Instead of the large-scale opening of the patient's chest to perform heart surgery, laparoscopic techniques have been used recently in which one or more small apertures are opened, and surgery is carried out by means of instruments controlled remotely by the surgeon, together with an observation device such as an endoscope. More recently, robotic procedures have been put into practice in laparoscopic surgery, in which the instruments inside the patient's body are not directly manipulated by the surgeon but are controlled by a computer-operated robotic apparatus which also may present a visual image of the operation site to the surgeon, for example the techniques developed by the companies Intuitive Surgical (known as Da Vinci) and Computer Motion.

In such laparoscopic techniques, it is necessary to deliver a surgically implantable construct containing viable cells to the implantation site, and manipulate it there. Hitherto this has been done by means of a surgical tool or tools having tweezer-like grippers. An implant construct such as a replacement blood vessel, which may be a blood vessel derived from the patient's own body or may be an artificial construct, is delicate and liable to damage. Its positioning in the body during its implantation, e.g. by suturing, requires care.

### SUMMARY OF THE INVENTION

The present inventors have noted a need for an improved delivery system for a delicate surgically implantable construct, particularly in laparoscopic surgery, for one or both of the steps of delivering the construct to the implantation site, and maintaining the construct in a desired position or positions at the site during the surgical operation.

According to the present invention there is provided a delivery assembly for use in surgery, comprising a surgically implantable construct containing viable cells, a support on which the construct is removably carried and holding means for releasably holding the construct on said support, wherein the support is selected from:

(i) a tube having an interior surface on which the construct is held, and

(ii) a rod having an exterior surface on which the construct is held.

This delivery assembly provides the advantages of protection of the construct, since it is held on the holding surface of the tube or rod in a controlled manner, and of making it possible to mount the construct in a predetermined and known position on the support, which is of value particularly in a robotic technique. The support can be easily moved and positioned and permits the construct to be released when desired. Surgical steps, such as suturing, can be carried out with the construct maintained in situ on the support. Techniques are available to the surgeon for suturing the construct, such as a blood vessel tube, even when it is mounted on the holding surface, such as the internal surface of a support in the form of a tube.

To provide a gentle holding of the construct, and easy application of the holding force and its release, the holding means is preferably suction holding means or one or more inflatable members. Suction holding means may comprise one or more apertures in the surface on which the construct is held. Such apertures can connect to a conduit or conduits within the support. The support surface may have one or more grooves connected to such an aperture or apertures. An inflatable member may form part of the support surface, and is expansible by means of gas pressure supplied through a conduit in the support in order to grip the construct. Alternatively or additionally an inflatable member may be separate from the support, for example a balloon-like member inserted within a tubular implantable construct.

The surgically implantable construct may be of natural origin, for example a blood vessel derived from the patient, or may be an artificial construct, in particular one containing viable cells which requires careful handling.

By viable cells are meant cells which are living or can be regarded as living since they are resuscitatable.

The artificial surgically implantable construct containing viable cells employed in this invention may be in the form of a sheet, tube or hollow organ and typically comprises a sheet, tube or hollow organ containing or consisting of conjoined cells and extracellular matrix, together with preferably a layer of endothelial cells on at least one surface thereof. The cells joined by extracellular matrix are typically smooth muscle cells or fibroblasts. Examples are

Sheet:

skin construct: sheet of fibroblasts and extracellular matrix (ECM), with an epithelial cell layer
   Hollow Organ (in the Form of Tube):
bladder: smooth muscle/ECM tube+transitional epithelium
stomach: smooth muscle/ECM+specialised gastric type epithelium
   Tube:
portion of gut (e.g. oesophagus, small intestine, colon, rectum):
   oesphagus—smooth muscle/ECM+squamous epithelium
   small intestine/colon/rectum—smooth muscle/ECM+columnar epithelium+goblet cells
   blood vessel or arterio-venous shunt: smooth muscle cells/ECM with endothelial cell layer
   urethra or ureter—smooth muscle/ECM+transitional epithelium

The production of artificial constructs in the form of sheets or tubes containing living cells, is described in our International Patent Application filed on 26 Mar. 2002 and published as WO 02/077336, and also in our co-pending UK Patent Application 0301834.8 filed 27 Jan. 2003. The content of these patent applications is incorporated herein by reference.

In the construct which is to be a portion of the gut, e.g. oesophagus, stomach, small intestine, colon or rectum, the smooth muscle cell tube may desirably have a multiple layer structure, e.g. double or triple layer. A method of forming such a multiple layer is disclosed in WO 02/077336.

Furthermore, the support of the delivery device of the invention may be a tube in which the artificial construct has been made in accordance with methods described in WO 02/077336.

To aid manipulation and positioning of the implantable construct, the support of the delivery assembly may be flexible in a manner such that it maintains a bent shape given to it. Certain plastics materials, e.g. in the form of a rod, hold a bent shape to which they are manipulated. Alternatively the support of the delivery assembly may be in the form of articulatedly connected elements, whose interconnections are adapted to maintain positions to which they are brought.

According to the invention there is also provided a method of surgery wherein a delivery assembly of the invention as described above is employed to bring the surgically implantable construct to its implantation site, and optionally hold it in position at said site during the performance of one or more surgical steps on it. In robotic surgery, the delivery assembly may be controlled in position by a computer controlled robotic device. Typically, the path by which the implantable construct is brought to the implantable site is outside the arterio-venous system of the patient. At the implantation site, the construct may be sutured while still supported by the support of the delivery device.

### BRIEF INTRODUCTION OF THE DRAWINGS

Embodiments of the invention will now be described by way of example, with reference to the accompanying drawings, in which:

FIG. 1 is a schematic sectional view of a delivery assembly of the invention.

FIG. 2 is a schematic sectional view of the second delivery assembly according to the invention.

### DESCRIPTION OF EMBODIMENTS

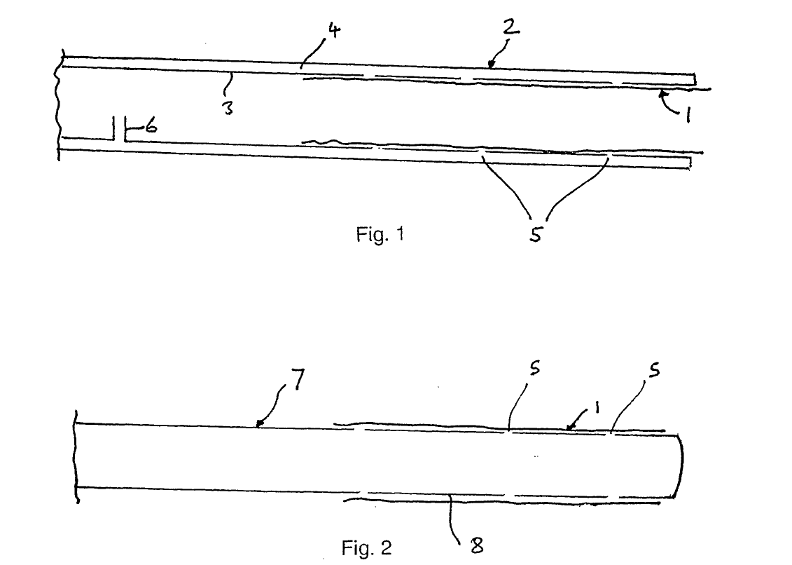

FIG. 1 shows an implantable surgical construct in the form of a flexible tube 1 which is to be implanted as a replacement blood vessel in the patient. As mentioned above, the implant contains or consists of living cells. The tube 1 is carried on the internal cylindrical surface 3 of a rigid cylindrical tubular support 2. The support has a hollow wall 4 and apertures 5 opening at the internal surface, so that by application of reduced pressure inside the wall 4, the implant tube 1 is held by suction against the surface 3. A connection conduit 6 is shown, for the application of the suction pressure. As shown, the implant tube 1 projects slightly from the support 2, so that its end is available for suturing at the surgical site, but this is not necessary. A typical diameter of the tube 1 is 0.5 cm.

An alternative form of holding means for the implant tube 1 in the assembly of FIG. 1 is a balloon (not shown) which is inserted inside the tube 1 and inflated to hold the tube 1 against the internal surface 3 of the support.

The support 2 can be held at its remote end (not shown) in order to be inserted into the patient through a laparoscopic aperture and can be readily manipulated, e.g. by robotic means. The holding and release of the implant tube 1 by the support is also controlled remotely.

FIG. 2 shows a second delivery assembly embodying the invention, in which the support for the surgical implant tube 1 is a hollow rigid rod 7 having apertures 5 in its surface for the application of suction pressure to hold the implant tube 1 on the external surface 8 of the rod 7. Although the implant tube 1 is exposed in this embodiment, it is nevertheless protected against kinking or other gross deformation by the supporting rod 7 and can be brought to the surgical site in a controlled manner and maintained accurately in a controlled position at the site, for example while suturing is carried out on it. Instead of the use of suction apertures for holding the implant tube 1 on the support surface 8, the rod may have one or more inflatable structures on its surface which are expanded by pressurised gas supplied along the hollow rod, to grip the implant tube 1. As in the case of the support tube of FIG. 1, the support rod is suitable to be held and controlled from the exterior of the patient's body, for example by robotic device.

1. Delivery assembly for use in surgery, comprising a surgically implantable construct containing viable cells, a support on which said construct is removably carried and holding means for releasably holding said construct on said support, wherein said support is selected from
(i) a tube having an interior surface on which said construct is held, and
(ii) a rod having an exterior surface on which said construct is held.

2. Delivery assembly according to claim 1, wherein said holding means is suction holding means comprising one or more apertures in said surface on which said construct is held.

3. Delivery assembly according to claim 1, wherein said holding means comprises one or more inflatable members inflatable to grip said construct.

4. Delivery assembly according to claim 1, wherein said surgically implantable construct is a tube.

5. Delivery assembly according to claim 1, wherein said surgically implantable construct is of natural origin.

6. Delivery assembly according to claim 1, wherein said surgically implantable construct is artificial.

7. Delivery assembly according to claim 1, wherein said support is flexible and adapted to maintain a bent shape to which it is brought.

8. A method of surgery wherein a delivery assembly according to claim 1 is employed to bring said construct to its implantation site and hold it in position at said site.

9. A method according to claim 8, wherein said delivery assembly is controlled in position by a computer-controlled robotic device.
